(19) United States
(12) Patent Application Publication
(10) Pub. No.: US 2024/0272153 A1
(43) Pub. Date: Aug. 15, 2024

(54) METHOD FOR RE-USING A TEST SOLID SURFACE IN A BIOCHEMICAL ASSAY

(71) Applicant: ACCESS MEDICAL SYSTEMS, LTD., Palo Alto, CA (US)

(72) Inventors: Haode Chen, Shanghai (CN); Robert F. Zuk, Menlo Park, CA (US)

(21) Appl. No.: 18/628,497

(22) Filed: Apr. 5, 2024

Related U.S. Application Data
(63) Continuation of application No. PCT/US2022/077625, filed on Oct. 5, 2022.
(60) Provisional application No. 63/262,317, filed on Oct. 8, 2021.

(51) Int. Cl. G01N 33/543; G01N 21/64; G01N 33/533

(52) U.S. Cl. CPC G01N 33/54393; G01N 21/6428; G01N 33/533

(57) ABSTRACT

The present invention is directed biochemical assay methods with regenerated substrate surface for immunoassay reactions. The methods clean a ceramic or glass substrate surface with atmospheric gas plasma and then coat the cleaned surface directly (without further modification of the cleaned surface) with a hapten-polymer conjugate, a protein, or an anti-non-human IgG antibody as a first layer on the substrate surface for subsequence assay reactions. After the completion of each cycle of reactions, the substrate is cleaned by the same plasma cleaning procedure, to elute the organic molecules formed on the substrate surface and to regenerate the substrate for another cycle of immunoassay reactions. The regeneration protocols can be repeated 1-10 times and maintains acceptable assay performance.

### CROSS-REFERENCE TO RELATED APPLICATION(S)

This application is a continuation of PCT/US2022/077625, filed Oct. 5, 2022; which claims the benefit of U.S. Provisional Application No. 63/262,317, filed Oct. 8, 2021. The contents of the above-identified applications are incorporated herein by reference in their entirety.

### TECHNICAL FIELD

The present invention related to a biochemical assay method, e.g., an immunoassay method, which regenerates and re-uses a solid surface for quantitating an analyte in different samples, from about 3 to 25 times. The method cleans a solid surface by plasma cleaning, which strips off all the protein layers bound on the surface during biochemical reactions, and allows the cleaned solid surface to be used immediately for a new biochemical assay.

### BACKGROUND OF THE INVENTION

Cost containment is a major goal for healthcare providers worldwide. In vitro diagnostics (IVD) is no exception, where the clinical utility of biomarkers in the diagnosis and prognosis has become standard in-patient management. Immunoassay technology is large portion of the IVD industry and is steadily growing, about 3%/year in the U.S. and 15-20%/year in developing countries. In some cases, such as serial measurements for cardiac markers in diagnosing myocardial infarction, cost can limit the appropriate amount of testing.

Typical approaches to reducing the cost of immunoassays entail minimizing manufacturing expenses for materials, labor, and facilities overhead.

Any method to recycle immune reagents typically centers upon disassociating the immune complex with a denaturing agent such as an acidic/basic pH solution, organic solvents, chaotropic agents, etc. The denaturation step changes the antibody charge, hydration, hydrogen bonding and tertiary structure where it no longer binds to antigen. Exposing the antibody back to the initial binding conditions close to physiologic pH and ionic strength, is expected to restore original binding activity, however, few antibodies can tolerate repeated exposures to denaturation conditions without adversely impacting some aspect of their binding properties and consequently, assay performance.

There is a need for reducing the cost of immunoassays, while maintaining the assay performance at the same time.

### BRIEF DESCRIPTION OF THE DRAWINGS

FIG. 1 illustrates an optical configuration, for detection of fluorescence on the probe tip.

FIG. 2 depicts a procalcitonin (PCT) assay configuration with a hapten (H)-BSA coated probe, a conjugate of anti-H/anti-PCT as capture antibody, Cy5-anti PCT as signal AB.

### DETAILED DESCRIPTION OF THE INVENTION

### Definitions

Terms used in the claims and specification are to be construed in accordance with their usual meaning as understood by one skilled in the art except and as defined as set forth below.

“About,” as used herein, refers to within +10% of the recited value.

An “analyte-binding” molecule, as used herein, refers to any molecule capable of participating in a specific binding reaction with an analyte molecule. Examples include but are not limited to, (i) antigen molecules, for use in detecting the presence of antibodies specific against that antigen; (ii) antibody molecules, for use in detecting the presence of antigens; (iii) protein molecules, for use in detecting the presence of a binding pair for that protein; (iv) ligands, for use in detecting the presence of a binding pair; or (v) single stranded nucleic acid molecules, for detecting the presence of nucleic acid binding molecules.

An “aspect ratio” of a shape refers to the ratio of its longer dimension to its shorter dimension.

A “binding molecular,” refers to a molecule that is capable to bind another molecule of interest.

“A binding pair,” as used herein, refers to two molecules that are attracted to each other and specifically bind to each other. Examples of binding pairs include, but not limited to, an antigen and an antibody against the antigen, a ligand and its receptor, complementary strands of nucleic acids, biotin and avidin, biotin and streptavidin, lectin and carbohydrates. Preferred binding pairs are biotin and streptavidin, biotin and avidin, fluorescein and anti-fluorescein, digioxigenin/anti-digioxigenin. Biotin and avidin, including biotin derivatives and avidin derivatives such as streptavidin, may be used as intermediate binding substances in assay protocols employing complex binding sequences. For example, antibodies may be labeled with biotin (“biotinylated”) and used to bind to a target substance previously immobilized on a solid phase surface. Fluorescent compositions according to the present invention employing an avidin or streptavidin may then be used to introduce the fluorescent label.

“Chemiluminescence,” as used herein, refers to the emission of energy with limited emission of luminescence, as the result of a chemical reaction. For example, when luminol reacts with hydrogen peroxide in the presence of a suitable catalyst, it produces 3-aminophthalate in an excited state, which emits light when it decays to a lower energy level.

“Immobilized,” as used herein, refers to reagents being fixed to a solid surface. When a reagent is immobilized to a solid surface, it is either be non-covalently bound or covalently bound to the surface.

A “monolithic substrate,” as used herein, refers to a single piece of a solid material such as glass, quartz, or plastic that has one refractive index.

“Plasma” is the electrical ionization of a gas. The plasma is created by using high frequency voltages (typically kHz to >MHz) to ionize a gas. The plasma discharge creates a smooth, undifferentiated cloud of ionized gas, which can react and degrade organic compounds. A plasma's activated species include atoms, molecules, ions, electrons, free radicals, metastables, and photons in the short-wave ultraviolet range. This mixture then interacts with any surface placed in the plasma. Plasma cleaning removes organics contamination through chemical reaction or physical ablation of hydrocarbons on treated surfaces.

A “probe,” as used herein, refers to a substrate coated with a thin-film layer of molecules at the sensing side; such molecules participate in binding during an assay. A probe has a distal end and a proximal end. The proximal end (also refers to probe tip in the application) has a sensing surface coated with a thin layer of analyte-binding molecules.

The present invention discloses a method to treat and re-use a biochemical assay substrate, from about 3 to 25 times, while maintaining acceptable assay performance. The present invention optionally re-use reagents and saves the cost per test.

The substrate is a solid substrate made of glass, ceramic, plastic, quartz, silicon, or a mixture thereof. The surface of the solid substrate is coated with a polymer (e.g., a protein), where the immunoassay reactions occur. The solid substrate can be in a form of a probe, a microwell, a disk, or a bead, a slide, or a plate. In one embodiment, the solid substrate is a probe.

The probe can be a monolithic substrate or an optical fiber. The probe can be any shape such as rod, cylindrical, round, square, triangle, etc., with an aspect ratio of length to width of at least 5 to 1, preferably 10 to 1. Because the probe is dipped in a sample solution and one or more assay solutions during an immunoassay, it is desirable to have a long probe with an aspect ratio of at least 5 to 1 to enable the probe tip's immersion into the solutions. The surface of the probe tip is coated with biomolecules and bound with fluorescent labels after immunoassay reactions. In a preferred embodiment, the probe tip surface has a small dimension (≤5 mm in diameter) so that there is negligible consumption of the signal reagent and amplification reagent, and no replenish of those reagents is necessary during the assay cycles.

### Fluorescent Detection System

The present invention uses a fluorescent detection system as described in U.S. Pat. No. 8,492,139, which is incorporated herein by reference, for measuring a fluorescent signal on a probe tip. The system comprises: (a) a probe having an aspect ratio of length to width at least 5 to 1, the probe having a first end and a second end, the second end having a sensing surface bound with a fluorescent label; (b) a light source for emitting excitation light directly to the probe's sensing surface; (c) a collecting lens pointed toward the sensing surface; and (d) an optical detector for detecting the emission fluorescent light; where the collecting lens collects and directs the emission fluorescent light to the optical detector.

Any light source that can emit proper excitation light for the fluorescent label is suitable for the present invention. A prefer light source is a laser that can emit light with wavelengths suitable for fluorescent labels. For example, the laser center wavelength is preferred to be 649 nm for Cy5 fluorescent dye. A suitable optical detector for detecting emission light is a photomultiplier tube (PMT), a charge coupled device (CCD), or a photodiode.

The light source and the optical detector including the collecting lens are mounted on the same side of the probe tip surface (the sensing surface). If the sensing surface faces down, they are both mounted below the tip surface. If the sensing surface faces up, they are both mounted above the tip surface. They are closer to the sensing surface than the other end of the probe. The sensing surface is always within the numeric aperture of the collecting lens. The probe can be, but it does not have to be centrally aligned with the collecting lens.

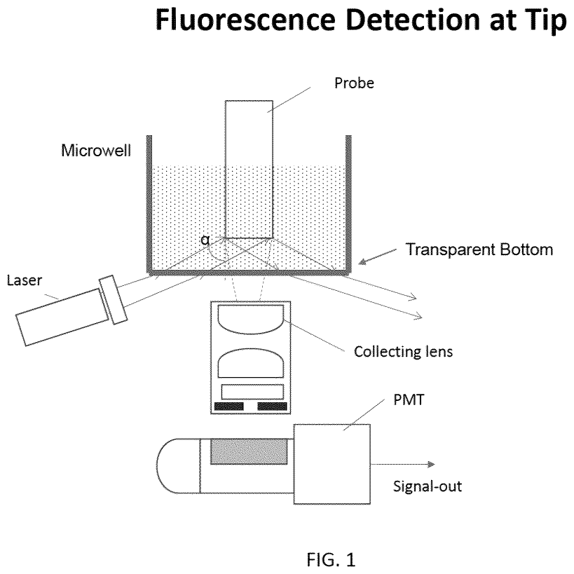

FIG. 1 illustrates an embodiment of the fluorescent detection system. Alternatively, the methods of the present invention can be detected by other suitable fluorescence detection systems or chemiluminescence detection systems.

### (I). Detecting an Analyte by a Recycling Protocol Using a Hapten-Coated Substrate

The present invention is directed to a method of detecting an analyte in multiple liquid samples, using the same solid substrate.

### First Embodiment

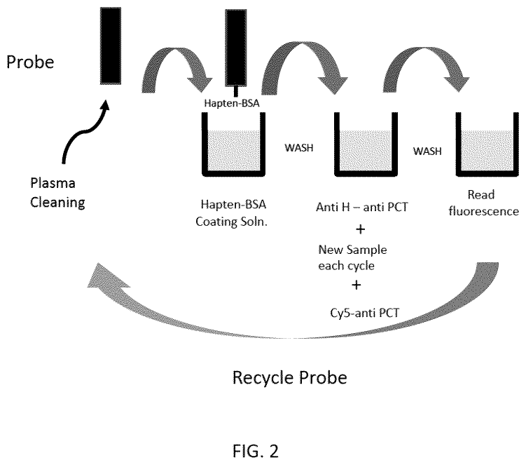

In a first embodiment, the method comprises the steps of: (a) cleaning a surface of a solid substrate by atmospheric gas plasma between 5 seconds to 5 minutes; (b) coating the cleaned surface directly with a hapten-polymer conjugate contained in an aqueous solution; (c) forming an immunocomplex comprising an analyte from a sample, a capture antibody, and a signal antibody on the coated surface of (b), wherein the capture antibody and the signal antibody are two different antibodies against two different epitopes the analyte, the capture antibody is covalently linked to an anti-hapten antibody against the hapten and the signal antibody is conjugated with fluorescent labels; (d) washing the surface with a wash solution; (c) determining the analyte concentration in the sample by measuring the fluorescent signal of the immunocomplex formed on the surface, and (f) repeating the steps (a)-(c) except in step (c) with an analyte from a new sample, whereby the analyte in each of the multiple liquid samples is detected. The assay protocol is illustrated in FIG. 2.

The solid substrate preferably is a probe that has a small tip for binding biomolecules. The tip has a smaller surface area with a diameter ≤5 mm, preferably ≤2 mm or ≤1 mm. The small surface of the probe tip endows it with several advantages. In solid phase immunoassays, having a small surface area is advantageous because it has less non-specific binding and thus produces a lower background signal. Further, the reagent or sample carry over on the probe tip is extremely small due to the small surface area of the tip. This feature makes the probe tip easy to wash, and it causes negligible contamination in the wash solution since the wash solution has a larger volume. Another aspect of the small surface area of the probe tip is that it has small binding capacity. Consequently, when the probe tip is immersed in a reagent solution, the binding of the reagent does not consume a significant amount of the reagent. The reagent concentration is effectively unchanged. Negligible contamination of the wash solution and small consumption of the reagents enable the reagents and the wash solution to be re-used many times, for example, 3-10 times or 3-20 times.

In step (a), the method uses atmospheric gas plasma to clean the substrate surface. Preferably, the atmospheric gas plasma is ionized by atmospheric pressure that approximately matches that of the surrounding atmosphere. Atmospheric pressure plasmas have advantages over low-pressure plasma or high-pressure plasma because no sealed reaction vessel is needed to maintain the pressure level differing from atmospheric pressure. The need for cost-intensive chambers for producing a partial vacuum as used in low-pressure plasma technology is eliminated. The substrate is exposed to room temperature atmospheric gas plasma at atmospheric pressure for 5 seconds to 1 minute, or 10 seconds to 2 minutes, or 15 seconds to 1 minute, to remove organic molecules on the solid surface. To enhance the cleaning effect, the plasma cleaning can be repeated 1, 2, or 3 times. For example, the plasma cleaning can be performed for 5-20 seconds, and repeated 1-3 times.

To enhance the cleaning effect, the substrate surface is optionally washed and cleaned with a protein removal solution or a protein denaturant solution for 5 seconds to 1 minute, or 10 seconds to 2 minutes, or 15 seconds to 1 minute, before the plasma cleaning. The protein removal solution generally comprises a strong acid with pH<3, a strong alkali with pH>11, high salts with >500 mM ionic strength, a chaotropic agent, a surfactant, or a combination thereof. For example, the protein removal solution is a strong acid such as 1-5 M or 1-3 M hydrochloric acid, sulfuric acid, nitric acid. For example, the protein removal solution is a strong alkali such 1-5 M NaOH. For example, the protein removal solution is a high salt such as 1-3 M NaCl or KCl. For example, the protein removal solution is a chaotropic agent such as dimethylsulfoxide (DMSO), n-butanol, ethanol, guanidinium chloride, lithium perchlorate, lithium acetate, magnesium chloride, phenol, 2-propanol, thiourea, and urea. For example, the protein removal solution is a surfactant or an ionic surfactant such as sodium dodecyl sulfate (SDS). For example, the protein removal solution comprises 1-3 M HCl and 1-3 M NaCl.

To enhance the cleaning effect, the solid-phase substrate can rotate at a velocity of 300-1,800 rpm to increase the interaction of the solid-phase substrate interface with the protein removal solution. Then the solid-phase substrate is exposed to room temperature plasma for deep cleaning. The protein removal solution cleaning and plasma cleaning can be repeated 1-5 times, for example 1-3 times.

In step (b), the cleaned surface of the solid substrate is coated directly with a hapten-polymer conjugate contained in an aqueous solution. The hapten-polymer is non-covalently adsorbed to the clean surface directly, without any physical or chemical modification of the cleaned surface first. For example, the cleaned surface is not coated with aminopropylsilane, aminopropyl tricthoxysilane, or any other molecule, before reacting with the hapten-polymer conjugate.

Suitable haptens for the present invention include, for example, small organic molecules such as nitrotyrosine, dinitrophenol, trinitrophenol, nitrophenol, and aminobenzoic acid; dyes such as TRITC, Lucifer Yellow, Texas Red, and Bodipy; peptides such as Myc, Flag, and polyhistidine; drugs such as theophylline, phenytoin, phenobarbital, valproic acid, penicillin, and gentamycin; steroids such as progesterone, testosterone, and estradiol, and vitamins such as biotin and Vitamin D. Preferred haptens are fluorescein, biotin, and digoxigenin.

The hapten is conjugated to a polymer to facilitate the adsorption to the clean surface. Suitable polymers include protein, DNA, polysaccharides, polyethylene glycol, liposomes, polylysine, polyacrylic acid, dendrimers, etc. Preferred polymer is a protein such as albumin, ovalbumin, IgG, fibronectin, thyroglobulin, etc. Conjugating a hapten to a polymer can be performed according to any procedure well-known to a skilled person in the art, e.g., sec WO2020/206175.

In step (c), an immunocomplex is formed on the coated surface. The immunocomplex comprises an analyte from a sample, a capture antibody, and a signal antibody.

In one embodiment, the immunocomplex of step (c) is formed by: (c1) mixing a sample solution with a dual antibody solution and a reagent solution to form a mixture, wherein the sample solution comprises the analyte, the dual antibody solution comprises the anti-hapten antibody covalently linked to the capture antibody, and the reagent solution comprises the signal antibody conjugated with fluorescent labels; and (c2) contacting the coated surface of (b) with the mixture of (c1) to form an immunocomplex on the surface.

In another embodiment, the immunocomplex of step (c) is formed by: (c1) contacting the coated surface of (b) with a dual antibody solution comprising the anti-hapten antibody covalently linked to the capture antibody; (c2) contacting the surface of (cl) with a sample solution comprising the sample; and (c3) contacting the surface of (c2) with a reagent solution comprising the signal antibody conjugated with fluorescent labels to form an immunocomplex on the surface.

In one embodiment, the anti-hapten antibody and the capture antibody are directly linked to each other without a linker.

In a preferred embodiment, the anti-hapten antibody and the capture antibody are both covalently linked to a polymer, which serves as a linker or spacer. The polymer in general has a molecular weight of 1,000 to 500,000 Daltons. The polymer can be a polysaccharide (e.g., dextran, amylose), a dendrimer, or a polyethylene glycol. In one preferred embodiment, the polymer is FICOLL® (copolymers of sucrose and epichlorohydrin).

The signal antibody is conjugated to fluorescent labels. Any suitable fluorescent label can be used in this method. For example, the fluorescent dye is Cy5 (molecule weight MW 792), Alexa Fluor 647, DyLight 350 (MW 874), DyLight 405 (MW793), DyLight 488 (MW 71011), DyLight 550 (MW 982), DyLight 594 (MW 1078), DyLight 633 (MW 1066), DyLight 650 (MW 1008), DyLight 680 (MW 950), DyLight 755 (MW 1092), DyLight 800 (MW 1050), an Oyster fluorescent dye, IRDye, or organic compounds comprising multiple rings chelated with a rare earth metal such as a lanthanide (Eu, Th, Sm, or Dy).

An example of a fluorescent label is an arylsulfonate cyanine fluorescent dye as described in Mujumdar et al. (1993) Bioconjugate Chemistry, 4: 105-111; Southwick et al. (1990) Cytometry, 11: 418-430; and U.S. Pat. No. 5,268,486. Cy5 is a preferred arylsulfonate cyanine fluorescent dye, because it has a high extinction coefficient and good quantum yield; it also has fluorescent emission spectra in a range (500 nm to 750 nm) outside of the auto-fluorescence wavelengths of most biological materials and plastics. In addition, Cy5 has a good solubility in water, and has low non-specific binding characteristics.

A fluorescent label can covalently bind to the signal antibody through a variety of moieties, including disulfide, hydroxyphenyl, amino, carboxyl, indole, or other functional groups, using conventional conjugation chemistry as described in the scientific and patent literature. Exemplary techniques for binding arylsulfonate cyanine fluorescent dye labels to antibodies and other proteins are described in U.S. Pat. Nos. 5,268,486; 5,650,334; the contents of which are in incorporated herein by reference. Techniques for linking a preferred Cy5 fluorescent label to antibodies are described in a technical bulletin identified as Cat. No. A25000, published by Biological Detection Systems, Inc., Pittsburgh, Pa.

In step (d), the solid surface is washed 1-5 times, preferably 1-3 times with a wash solution. The wash solution typically contains buffer and a surfactant such as Tween 20. When the solid substrate is a probe, the probe is dipped in a wash vessel containing a wash solution for washing.

In step (e), the analyte concentration is determined by measuring the fluorescent signal of the immunocomplex formed on the surface. When the solid substrate is a probe, the probe stays in the wash vessel or is moved to a measurement vessel and the fluorescent signal of the bound immunocomplex is detected by the fluorescent detection system as described above.

The analyte concentration in the sample is determined by measuring the fluorescent signal of the immunocomplex at the probe tip, optionally subtracting a pre-read fluorescent signal of the solid surface, and quantitating against a calibration curve (a standard curve).

The calibration curve is typically pre-established before assaying the samples according to the methods known to a person skilled in the art. In a preferred embodiment, the fluorescent signals of the same sample remain constant at each cycle, and the calibration curves are the same for each cycle.

### Second Embodiment

In a second embodiment, the method includes an amplification step with a second hapten and anti-second hapten antibody or streptavidin. The method amplifies the fluorescent signals with a second hapten and an anti-second hapten antibody or streptavidin. The second hapten is different from the hapten immobilized on the probe tip. The details of each step are the same or similar to those of the corresponding steps described above in the first embodiment above.

The method comprises the steps of: (a) cleaning a ceramic or glass surface by atmospheric gas plasma between 5 seconds to 5 minutes; (b) coating the cleaned surface directly with a hapten-polymer conjugate contained in an aqueous solution; (c) forming a first immunocomplex comprising an analyte from a sample, a capture antibody, and a signal antibody on the coated surface of (b), wherein the capture antibody and the signal antibody are two different antibodies against two different epitopes of the analyte, the capture antibody is covalently linked to an anti-first hapten antibody against the first hapten and the signal antibody is conjugated with a second hapten, the first hapten and the second hapten are different; (d) washing the surface with a wash solution; (e) contacting the surface with an amplification solution comprising an anti-second hapten antibody or streptavidin conjugated to fluorescent labels to form a second immunocomplex comprising the analyte, the capture antibody, the signal antibody, the second hapten, and the anti-second hapten antibody or streptavidin on the probe tip; (f) determining the analyte concentration in the sample by measuring the fluorescent signal of the second immunocomplex formed on the surface, and (g) repeating the steps (a)-(f) except in step (c) with an analyte from a new sample, whereby the analyte in each of the multiple liquid samples is detected.

In one embodiment, the first immunocomplex of step (c) is formed by: (c1) mixing a sample solution with a dual antibody solution and a reagent solution to form a mixture, wherein the sample solution comprises the analyte, the dual antibody solution comprises the anti-first hapten antibody covalently linked to the capture antibody, and the reagent solution comprises the signal antibody conjugated to the second hapten; and (c2) contacting the coated surface of (b) with the mixture of (c1) to form the first immunocomplex on the surface.

In another embodiment, the immunocomplex of step (c) is formed by: (c1) contacting the coated surface of (b) with a dual antibody solution comprising the anti-first hapten antibody covalently linked to the capture antibody; (c2) contacting the surface of (c1) with a sample solution comprising the sample; and (c3) contacting the surface of (c2) with a reagent solution comprising the signal antibody conjugated with the second hapten to form the first immunocomplex on the surface.

The detail of each step is similar to the corresponding steps described above in (I), first embodiment.

### (II). Detecting an Analyte by a Recycling Protocol Using a First Member of a Binding Pair-Coated Substrate

The present invention is also directed to a method of detecting an analyte in multiple liquid samples, using a regenerated solid substrate coated with a protein that is a first member of a binding pair.

A binding pair is defined in the Definitions. For example, a protein that is a first member of a binding pair is anti-hapten antibody or streptavidin or avidin, and a second member of a binding pair is a corresponding hapten such as fluorescein, DNP, Digoxigennin, estradiol, T4, or biotin. For example, a first member of a binding pair is a protein antigen and the second member of a binding pair is its corresponding antibody.

The details of each step are the same or similar to those of the corresponding steps described above in (I), first embodiment.

### First Embodiment

In the first embodiment, the method comprises the steps in the order of: (a) cleaning a ceramic or glass surface by atmospheric gas plasma between 5 seconds to 5 minutes; (b) coating the cleaned surface directly with a protein that is a first member of a binding pair contained in an aqueous solution; (c) forming an immunocomplex comprising an analyte from a sample, a capture antibody, and a signal antibody on the coated surface of (b), wherein the capture antibody and the signal antibody are two different antibodies against two different epitopes the analyte, the capture antibody is covalently linked to a second member of a binding pair and the signal antibody is conjugated with fluorescent labels; (d) washing the surface with a wash solution; (e) determining the analyte concentration in the sample by measuring the fluorescent signal of the immunocomplex formed on the surface, and (f) repeating the steps (a)-(e) except in step (c) with an analyte from a new sample, whereby the analyte in each of the multiple liquid samples is detected.

In one embodiment, the immunocomplex of step (c) is formed by: (c1) mixing a sample solution with a dual conjugate solution and a reagent solution to form a mixture, wherein the sample solution comprises the analyte, the dual conjugate solution comprises the capture antibody covalently linked to the second member of a binding pair, and the reagent solution comprises the signal antibody conjugated with fluorescent labels; and (c2) contacting the coated surface of (b) with the mixture of (c1) to form an immunocomplex on the probe tip.

In another embodiment, the immunocomplex of step (c) is formed by: (c1) contacting the coated surface of (b) with a dual conjugate solution comprises the capture antibody covalently linked to the second member of a binding pair covalently linked to the capture antibody; (c2) contacting the surface of (c1) with a sample solution comprising the sample; and (c3) contacting the surface of (c2) with a reagent solution comprising the signal antibody conjugated with fluorescent labels to form an immunocomplex on the probe tip.

### Second Embodiment

In a second embodiment, the method includes an amplification step with a hapten (e.g., biotin) and an anti-hapten antibody or streptavidin. The method amplifies the fluorescent signals with a hapten and an anti-hapten antibody or streptavidin, conjugated to a fluorescent signal. This hapten is different from a first hapten, if used as a second member of the binding pair.

The method comprising the steps in the order of: (a) cleaning a ceramic or glass surface by atmospheric gas plasma between 5 seconds to 5 minutes; (b) coating the cleaned surface directly with a hapten-polymer conjugate contained in an aqueous solution; (c) forming a first immunocomplex comprising an analyte from a sample, a capture antibody, and a signal antibody on the coated surface of (b), wherein the capture antibody and the signal antibody are two different antibodies against two different epitopes of the analyte, the capture antibody is covalently linked to an anti-first hapten antibody against the first hapten and the signal antibody is conjugated with a second hapten, the first hapten and the second hapten are different; (d) washing the surface with a wash solution; (c) contacting the surface with an amplification solution comprising an anti-second hapten antibody or streptavidin conjugated to fluorescent labels to form a second immunocomplex comprising the analyte, the capture antibody, the signal antibody, the second hapten, and the anti-second hapten antibody or streptavidin on the probe tip; (f) determining the analyte concentration in the sample by measuring the fluorescent signal of the second immunocomplex formed on the surface, and (g) repeating the steps (a)-(f) except in step (c) with an analyte from a new sample, whereby the analyte in each of the multiple liquid samples is detected. When the second hapten is a biotin, streptavidin is used in step (e).

In one embodiment, the immunocomplex of step (c) is formed by: (c1) mixing a sample solution with a dual conjugate solution and a reagent solution to form a mixture, wherein the sample solution comprises the analyte, the dual conjugate solution comprises the capture antibody covalently linked to the second member of a binding pair, and the reagent solution comprises the signal antibody conjugated with fluorescent labels; and (c2) contacting the coated surface of (b) with the mixture of (c1) to form an immunocomplex on the probe tip.

In another embodiment, the immunocomplex of step (c) is formed by: (c1) contacting the coated surface of (b) with a dual conjugate solution comprises the capture antibody covalently linked to the second member of a binding pair covalently linked to the capture antibody; (c2) contacting the surface of (c1) with a sample solution comprising the sample; and (c3) contacting the surface of (c2) with a reagent solution comprising the signal antibody conjugated with fluorescent labels to form an immunocomplex on the probe tip.

### (III). Detecting an Analyte by a Recycling Protocol Using an Anti-Non-Human IgG-Coated Substrate

The present invention is further directed to a method of detecting an analyte in multiple liquid human samples, using a regenerated solid substrate coated with an anti-non-human IgG antibody or an antigen-binding fragment thereof (such as Fab, (Fab′)₂, or scFv). The non-human is mouse, rabbit, goat, sheep, or camel, and the anti-non-human IgG antibody has a minimal (e.g., <5%, <2%, <1% , or <0.5%) cross reactivity against human IgG,

The details of each step are the same or similar to those of the corresponding steps described above in (I), first embodiment.

### First Embodiment

In the first embodiment, the method comprises the steps in the order of: (a) cleaning a ceramic or glass surface by atmospheric gas plasma between 5 seconds to 5 minutes; (b) coating the cleaned surface directly with an anti-non-human IgG antibody or an antigen-binding fragment thereof contained in an aqueous solution; (c) forming an immunocomplex comprising an analyte from a sample, a capture antibody, and a signal antibody on the coated surface of (b), wherein the capture antibody and the signal antibody are two different antibodies against different epitopes of the analyte, the capture antibody is obtained from the non-human corresponding to the anti-non-human IgG, and the signal antibody is conjugated with fluorescent labels; (d) washing the surface with a wash solution; (e) determining the analyte concentration in the sample by measuring the fluorescent signal of the immunocomplex formed on the surface, and (f) repeating the steps (a)-(c) except in step (c) with an analyte from a new sample, whereby the analyte in each of the multiple liquid samples is detected.

In one embodiment, the immunocomplex of step (c) is formed by:(c1) mixing a sample solution with a capture antibody solution comprising the capture antibody and a reagent solution to form a mixture, wherein the sample solution comprises the analyte, and the reagent solution comprises the signal antibody conjugated with fluorescent labels; and (c2) contacting the coated surface of (b) with the mixture of (c1) to form an immunocomplex on the surface.

In another embodiment, wherein the immunocomplex of step (c) is formed by: (c1) contacting the coated surface of (b) with a capture antibody solution comprising the capture antibody; (c2) contacting the surface of (c1) with a sample solution comprising the sample; (b3) contacting the surface of (c2) with a reagent solution comprising the signal antibody conjugated with fluorescent labels to form an immunocomplex on the surface.

### Second Embodiment

In a second embodiment, the method includes an amplification step with a hapten and an anti-hapten antibody or streptavidin when the hapten is biotin. The method amplifies the fluorescent signals with a hapten and an anti-hapten antibody or streptavidin, conjugated to a fluorescent signal.

In one embodiment, the immunocomplex of step (c) is formed by: (c1) mixing a sample solution with a capture antibody solution comprising the capture antibody and a reagent solution to form a mixture, wherein the sample solution comprises the analyte, and the reagent solution comprises the signal antibody conjugated to the hapten; and (c2) contacting the coated surface of (b) with the mixture of (c1) to form the first immunocomplex on the surface.

In another embodiment, the immunocomplex of step (c) is formed by: (c1) contacting the coated surface of (b) with a capture antibody solution comprising the capture antibody; (c2) contacting the surface of (c1) with a sample solution comprising the sample; and (c3) contacting the surface of (c2) with a reagent solution comprising the signal antibody conjugated to the hapten to form the first immunocomplex on the surface.

Although the method described above uses fluorescent signal for detection, the method can easily replace a fluorescent label with a chemiluminescent label and uses chemiluminescent signal for detection.

Chemiluminescent label may be selected from the group consisting of: Ruthenium(II)tris-bipyridine (MW 1057), acridinium ester (9[[4-[3-[(2,5-dioxo-1-pyrrolidinyl)oxy]-3-oxopropyl]phenoxy]carbonyl]-10-methyl-acridinium trifluoromethane sulfonate, MW 632), and hemin (MW 652).

The invention is illustrated further by the following examples that are not to be construed as limiting the invention in scope to the specific procedures described in them.

### EXAMPLES

### Example 1: Hapten and Fluorescent Dye Labeling

Fluorescein (F) was labeled to BSA by a standard method. Biotin (B) was linked by a standard method to anti-procalcitonin (PCT), from Hytest Ltd. (see WO2020/206175).

### Example 2: Antibody-Antibody Conjugation

Linkage of anti-F (Jackson Immunoresearch) to anti-PCT followed the procedures described in WO2020/206175.

### Example 3: Preparation of Cy5-Streptavidin-Crosslinked FICOLL®

Cy5-streptavidin-crosslinked FICOLL® was prepared according to the procedures described in WO2020/206175.

### Example 4: Plasma Cleaning of Probe

Quartz probes, 1 mm diameter and 2 cm in length, were used in the experiment. A probe was put in a microwell, then 200 μL liquid cleaning solution containing 1M HCl and 1M NaCl was added to the well. After the cleaning solution is removed, room temperature atmospheric gas plasma is turned on to clean the probe. The process is repeated 3 times.

The room temperature plasma device is equipped with a removable gas filter, a compact CERAPLAS™ plasma generator, and an active fan. The process is repeated 3 times.

### Example 5: Recycle Immunoassays

Reagents and samples were assayed in a microwell at 120 μL using PHS pH 7.4, 0.05% Tween 20, 5 mg/ml as buffer with 40 μL mineral oil added. Wash buffer was PBS pH 7.4, 0.05% Tween 20 (PBST) at 200 μL. The probe was held stationary with the microwells stationed on an orbital shaker platform having a 1 mm diameter stroke. Orbital flow was used to accelerate binding kinetics.

### Example 6

### Assay Protocol of PCT (Procalcitonin)

The assay protocol is shown below.
1. Put quartz probe in a microwell. Wash probe with a cleaning solution (1M HCl and 1M NaCl. for 5 s at 600 rpm;
2. Remove probe for plasma cleaning: 10 s, 600 rpm;
3. Repeated 1 and 2 two times, then put cleaned probe in a microwell;
4. Add 1 mg/mL BSA-fluorescein conjugate in PBST (phosphate-buffer saline with 0.05% Tween): 120 s, 600 rpm;
5. Wash with PBST: 3 s, 600 rpm;
6. Add 20 μg/mL of dual antibody conjugate (anti-PCT and anti-fluorescein.: 180 s, 600 rpm;
7. Wash with PBST: 3 s, 600 rpm;
8. Add 0.5% BSA in PBST: 60 s, 600 rpm;
9. Wash with PBST: 3 s, 600 rpm;
10. Dilute PCT Sample 1: 3 and incubate for 120 s, 600 rpm;
11. Wash with PBST: 3s, 600 rpm;
12. Add 3 μg/mL biotin-anti-PCT antibody in PBST: 6 0s, 600rpm;
13. Wash with PBST: 3 s, 600 rpm;
14. Pre-read CY5 background fluorescent signal;
15. Add 5 μg/mL Cy5-streptavidin-FICOLL in PBST: 30 s, 600 rpm;
16. Wash with PBST: 3 s, 600 rpm;
17. Read CY5 signal;
18. Repeat steps 1 to 17 with a new sample at step 10.

### Experiment A

Samples containing either 100 or 0 ng/ml PCT were used at different cycles and tested for their fluorescent signals according to the above protocols. Samples containing 100 ng/mL PCT were used in cycles 1, 3, 5, 7, and 9. Samples containing 0 ng/ml PCT were used in cycles 2, 4, 6, 8, and 10. The fluorescent signals are shown in Table 1 below. The results show that the fluorescent signals of samples having 100 ng/mL PCT concentrations were consistent after multiple regeneration cycles and the background (0 ng/ml) remained low after multiple regeneration cycles.

TABLE 1

| | PCT concentration | Fluorescent |
|---|---|---|
| Cycle | (ng/mL) | Signal |
| 1 | 100 | 10978 |
| 2 | 0 | 24 |
| 3 | 100 | 11500 |
| 4 | 0 | 24 |
| 5 | 100 | 10134 |
| 6 | 0 | 23 |
| 7 | 100 | 11203 |
| 8 | 0 | 23 |
| 9 | 100 | 10786 |
| 10 | 0 | 25 |

### Experiment B

Ten different PCT samples were tested either with regenerated probes at the indicated cycles, or with fresh plasma cleaned probes. After step 17, each fluorescent signal was read against a calibration curve to determine the PCT concentration. The quantitated results in terms of ng/ml of PCT are shown in Table 2. The results show that the quantitation is in general consistent between fresh plasma cleaned probes and regenerated probes at different cycles.

| | PCT Concentration (ng/mL) | PCT Concentration (ng/mL) |
|---|---|---|
| Sample and | Use Regenerated Probe at | Use Fresh Plasma Cleaned |
| Cycle No. | different Cycle | Probe |
| 1 | 0.355 | 0.309 |
| 2 | 1.982 | 1.811 |
| 4 | 6.057 | 6.295 |
| 5 | 1.727 | 1.941 |
| 7 | 4.175 | 3.11 |
| 8 | 3.365 | 4.408 |
| 9 | 0.06 | 0.051 |
| 10 | 0.117 | 0.050 |

* Sample 3 tested at Cycle 3 and Sample 6 tested at Cycle 6 had higher concentrations of PCT beyond the calibration curve, and thus results are not shown here.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method of detecting an analyte in multiple liquid samples comprising an analyte, comprising the steps in the order of:
(a) cleaning a ceramic or glass surface by atmospheric gas plasma between 5 seconds to 5 minutes;
(b) coating the cleaned surface directly with a hapten-polymer conjugate contained in an aqueous solution;
(c) forming an immunocomplex comprising an analyte from a sample, a capture antibody, and a signal antibody on the coated surface of (b), wherein the capture antibody and the signal antibody are two different antibodies against two different epitopes the analyte, the capture antibody is covalently linked to an anti-hapten antibody against the hapten and the signal antibody is conjugated with fluorescent labels;
(d) washing the surface with a wash solution;
(e) determining the analyte concentration in the sample by measuring the fluorescent signal of the immunocomplex formed on the surface; and
(f) repeating the steps (a)-(e) except in step (c) with an analyte from a new sample, whereby the analyte in each of the multiple liquid samples is detected.

2. The method of claim 1, wherein the immunocomplex of step (c) is formed by:
(c1) mixing a sample solution with a dual antibody solution and a reagent solution to form a mixture, wherein the sample solution comprises the analyte, the dual antibody solution comprises the anti-hapten antibody covalently linked to the capture antibody, and the reagent solution comprises the signal antibody conjugated with fluorescent labels; and
(c2) contacting the coated surface of (b) with the mixture of (c1) to form an immunocomplex on the surface.

3. The method of claim 1, wherein the immunocomplex of step (c) is formed by:
(c1) contacting the coated surface of (b) with a dual antibody solution comprising the anti-hapten antibody covalently linked to the capture antibody;
(c2) contacting the surface of (c1) with a sample solution comprising the sample;
(b3) contacting the surface of (c2) with a reagent solution comprising the signal antibody conjugated with fluorescent labels to form an immunocomplex on the surface.

4. A method of detecting an analyte in multiple liquid samples comprising an analyte, comprising the steps in the order of:
(a) cleaning a ceramic or glass surface by atmospheric gas plasma between 5 seconds to 5 minutes;
(b) coating the cleaned surface directly with a hapten-polymer conjugate contained in an aqueous solution;
(c) forming a first immunocomplex comprising an analyte from a sample, a capture antibody, and a signal antibody on the coated surface of (b), wherein the capture antibody and the signal antibody are two different antibodies against two different epitopes of the analyte, the capture antibody is covalently linked to an anti-first hapten antibody against the first hapten and the signal antibody is conjugated with a second hapten, the first hapten and the second hapten are different;
(d) washing the surface with a wash solution;
(e) contacting the surface with an amplification solution comprising an anti-second hapten antibody or streptavidin conjugated to fluorescent labels to form a second immunocomplex comprising the analyte, the capture antibody, the signal antibody, the second hapten, and the anti-second hapten antibody or streptavidin on the probe tip;
(f) determining the analyte concentration in the sample by measuring the fluorescent signal of the second immunocomplex formed on the surface; and
(g) repeating the steps (a)-(f) except in step (c) with an analyte from a new sample, whereby the analyte in each of the multiple liquid samples is detected.

5. The method of claim 4, wherein the first immunocomplex of step (c) is formed by:
(c1) mixing a sample solution with a dual antibody solution and a reagent solution to form a mixture, wherein the sample solution comprises the analyte, the dual antibody solution comprises the anti-first hapten antibody covalently linked to the capture antibody, and the reagent solution comprises the signal antibody conjugated to the second hapten; and
(c2) contacting the coated surface of (b) with the mixture of (c1) to form the first immunocomplex on the surface.

6. The method of claim 4, wherein the immunocomplex of step (c) is formed by:
(c1) contacting the coated surface of (b) with a dual antibody solution comprising the anti-first hapten antibody covalently linked to the capture antibody;
(c2) contacting the surface of (c1) with a sample solution comprising the sample; and
(c3) contacting the surface of (c2) with a reagent solution comprising the signal antibody conjugated with the second hapten to form the first immunocomplex on the surface.

7. A method of detecting an analyte in multiple liquid samples comprising an analyte, comprising the steps in the order of:
(a) cleaning a ceramic or glass surface by atmospheric gas plasma between 5 seconds to 5 minutes;
(b) coating the cleaned surface directly with a protein that is a first member of a binding pair contained in an aqueous solution;
(c) forming an immunocomplex comprising an analyte from a sample, a capture antibody, and a signal antibody on the coated surface of (b), wherein the capture antibody and the signal antibody are two different antibodies against two different epitopes the analyte, the capture antibody is covalently linked to a second member of a binding pair and the signal antibody is conjugated with fluorescent labels;
(d) washing the surface with a wash solution;
(e) determining the analyte concentration in the sample by measuring the fluorescent signal of the immunocomplex formed on the surface; and
(f) repeating the steps (a)-(e) except in step (c) with an analyte from a new sample, whereby the analyte in each of the multiple liquid samples is detected.

8. The method of claim 7, wherein the immunocomplex of step (c) is formed by:
(c1) mixing a sample solution with a dual conjugate solution and a reagent solution to form a mixture, wherein the sample solution comprises the analyte, the dual conjugate solution comprises the capture antibody covalently linked to the second member of a binding pair, and the reagent solution comprises the signal antibody conjugated with fluorescent labels; and
(c2) contacting the coated surface of (b) with the mixture of (c1) to form an immunocomplex on the probe tip.

9. The method of claim 7, wherein the immunocomplex of step (c) is formed by:
(c1) contacting the coated surface of (b) with a dual conjugate solution comprises the capture antibody covalently linked to the second member of a binding pair covalently linked to the capture antibody;
(c2) contacting the surface of (c1) with a sample solution comprising the sample;
(c3) contacting the surface of (c2) with a reagent solution comprising the signal antibody conjugated with fluorescent labels to form an immunocomplex on the probe tip.

10. A method of detecting an analyte in multiple liquid samples comprising an analyte, comprising the steps in the order of:
(a) cleaning a ceramic or glass surface by atmospheric gas plasma between 5 seconds to 5 minutes;
(b) coating the cleaned surface directly with a protein that is a first member of a binding pair contained in an aqueous solution;
(c) forming a first immunocomplex comprising an analyte from a sample, a capture antibody, and a signal antibody on the coated surface of (b), wherein the capture antibody and the signal antibody are two different antibodies against two different epitopes of the analyte, the capture antibody is covalently linked to a second member of a binding pair and the signal antibody is conjugated with a hapten;
(d) washing the surface with a wash solution;
(e) contacting the surface with an amplification solution comprising an anti-hapten antibody or streptavidin conjugated to fluorescent labels to form a second immunocomplex comprising the analyte, the capture antibody, the signal antibody, the hapten, and the anti-hapten antibody or streptavidin on the surface;
(f) determining the analyte concentration in the sample by measuring the fluorescent signal of the second immunocomplex formed on the surface; and
(g) repeating the steps (a)-(f) except in step (c) with an analyte from a new sample, whereby the analyte in each of the multiple liquid samples is detected.

11. The method of claim 10, wherein the immunocomplex of step (c) is formed by:
(c1) mixing a sample solution with a dual conjugate solution and a reagent solution to form a mixture, wherein the sample solution comprises the analyte, the dual conjugate solution comprises the captured antibody covalently linked to the second member of a binding pair, and the reagent solution comprises the signal antibody conjugated to a hapten; and
(c2) contacting the coated surface of (b) with the mixture of (c1) to form the first immunocomplex on the surface.

12. The method of claim 10, wherein the immunocomplex of step (c) is formed by:
(c1) contacting the coated surface of (b) with a dual conjugate solution comprising the second member of a binding pair covalently linked to the capture antibody;
(c2) contacting the surface of (c1) with a sample solution comprising the sample; and
(c3) contacting the surface of (c2) with a reagent solution comprising the signal antibody conjugated to a hapten to form a first immunocomplex on the surface.

13. A method of detecting an analyte in multiple liquid human samples comprising an analyte, comprising the steps in the order of:
(a) cleaning a ceramic or glass surface by atmospheric gas plasma between 5 seconds to 5 minutes;
(b) coating the cleaned surface directly with an anti-non-human IgG antibody or an antigen-binding fragment thereof contained in an aqueous solution;
(c) forming an immunocomplex comprising an analyte from a sample, a capture antibody, and a signal antibody on the coated surface of (b), wherein the capture antibody and the signal antibody are two different antibodies against different epitopes of the analyte, the capture antibody is obtained from the non-human corresponding to the anti-non-human IgG, and the signal antibody is conjugated with fluorescent labels;
(d) washing the surface with a wash solution;
(e) determining the analyte concentration in the sample by measuring the fluorescent signal of the immunocomplex formed on the surface, and (f) repeating the steps (a)-(e) except in step (c) with an analyte from a new sample, whereby the analyte in each of the multiple liquid samples is detected.

14. The method of claim 13, wherein the immunocomplex of step (c) is formed by:
(c1) mixing a sample solution with a capture antibody solution comprising the capture antibody and a reagent solution to form a mixture, wherein the sample solution comprises the analyte, and the reagent solution comprises the signal antibody conjugated with fluorescent labels; and
(c2) contacting the coated surface of (b) with the mixture of (c1) to form the first immunocomplex on the surface.

15. The method of claim 13, wherein the immunocomplex of step (c) is formed by:
(c1) contacting the coated surface of (b) with a capture antibody solution comprising the capture antibody;
(c2) contacting the surface of (c1) with a sample solution comprising the sample;
(c3) contacting the surface of (c2) with a reagent solution comprising the signal antibody conjugated with fluorescent labels to form the first immunocomplex on the surface.

16. A method of detecting an analyte in multiple liquid samples comprising an analyte, comprising the steps in the order of:
(a) cleaning a ceramic or glass surface by atmospheric gas plasma between 5 seconds to 5 minutes;
(b) coating the cleaned surface directly with an anti-non-human IgG antibody or an antigen-binding fragment thereof contained in an aqueous solution;
(c) forming a first immunocomplex comprising an analyte from a sample, a capture antibody, and a signal antibody on the coated surface of (b), wherein the capture antibody and the signal antibody are two different antibodies against two different epitopes of the analyte, the capture antibody is obtained from the non-human corresponding to the anti-non-human IgG, and the signal antibody is conjugated with a hapten;
(d) washing the surface with a wash solution;
(e) contacting the surface with an amplification solution comprising an anti-hapten antibody or streptavidin conjugated to fluorescent labels to form a second immunocomplex comprising the analyte, the capture antibody, the signal antibody, the hapten, and the anti-hapten antibody or streptavidin on the probe tip;
(f) determining the analyte concentration in the sample by measuring the fluorescent signal of the second immunocomplex formed on the surface; and
(g) repeating the steps (a)-(f) except in step (c) with an analyte from a new sample, whereby the analyte in each of the multiple liquid samples is detected.

17. The method of claim 16, wherein the immunocomplex of step (c) is formed by:
(c1) mixing a sample solution with a capture antibody solution comprising the capture antibody and a reagent solution to form a mixture, wherein the sample solution comprises the analyte, and the reagent solution comprises the signal antibody conjugated to the hapten; and
(c2) contacting the coated surface of (b) with the mixture of (c1) to form the first immunocomplex on the surface.

18. The method of claim 16, wherein the immunocomplex of step (c) is formed by:
(c1) contacting the coated surface of (b) with a capture antibody solution comprising the capture antibody;
(c2) contacting the surface of (c1) with a sample solution comprising the sample; and
(c3) contacting the surface of (c2) with a reagent solution comprising the signal antibody conjugated to the hapten to form the first immunocomplex on the surface.

19. The method of claim 1, where step (a) further comprises washing the surface with a solution prior to cleaning by atmospheric gas plasma, wherein the solution is an acidic solution having pH<3, an alkali solution having pH>11, a high salt solution having >500 mM ionic strength, a chaotropic agent, a surfactant, or a combination thereof.

20. The method of claim 19, wherein the solution comprises 1-3 M HCl and 1-3 M NaCl.

21. The method of claim 1, wherein the atmospheric gas plasma is ionized by atmospheric pressure.
